(19) United States
(12) Patent Application Publication
(10) Pub. No.: US 2025/0292884 A1
(43) Pub. Date: Sep. 18, 2025

(54) SYSTEMS AND METHODS FOR INCENTIVIZING HEALTH-BASED INTERVENTIONS BASED ON INFLUENCE IN A DIGITAL COMMUNICATION NETWORK

(71) Applicant: Sequelae, Inc., Minneapolis, MN (US)

(72) Inventors: Sara Taylor, Edina, MN (US); Scott Archibald, Wayzata, MN (US); Mike Christy, Shakopee, MN (US); Natalie Sheils, St. Paul, MN (US); Steven J. Catani, Athens, GA (US)

(73) Assignee: Sequelae, Inc., Minneapolis, MN (US)

(21) Appl. No.: 19/073,684

(22) Filed: Mar. 7, 2025

Related U.S. Application Data
(60) Provisional application No. 63/565,597, filed on Mar. 15, 2024.

(51) Int. Cl. G16H 20/00; G06Q 30/0207; G16H 80/00

(52) U.S. Cl. CPC G16H 20/00; G06Q 30/0207; G16H 80/00

(57) ABSTRACT

A method to incentivizing health related activity based on social media presence is described. The method includes providing a digital communication network for members of a population to communicate with each other; and analyzing, by the digital communication network, the communications in the digital communication network to identify a user who has influence over other members. Then, the identified user is targeted for at least one intervention. The methods may also include providing digital communications of health-related activity based on incentives. A member of the population may be informed about incentives for sharing their experience. The system determines whether the member shares their experience, and, if they do, the system provides the incentive to the member.

### CROSS-REFERENCE TO RELATED APPLICATIONS

This application claims the benefit of U.S. Application No. 63/565,597, filed on Mar. 15, 2024, which is incorporated herein by reference in its entirety.

### BACKGROUND

Various embodiments relate generally to health care systems, methods, devices and computer programs and, more specifically, relate to incentivizing health related activity or interventions based on influence in a digital communication network.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Computers have changed the way people interact. Digital networks, which may include the use of social media, allow individuals to interact with others online and connect many people with a community. The online community can be utilized to help build positive behaviors and encourage people to make improvements in their lives.

What is needed is a way to build upon the use of digital communication networks in order to incentivizing health related activity and interventions based on influence in a digital communication network and/or social media presence.

### SUMMARY

Example aspects of the present disclosure include:

A method to provide an intervention in a subset of a population according to at least one embodiment of the present disclosure comprises providing a digital communication network (DCN) for members of a population to communicate with each other; analyzing communications in the DCN between the members; identifying the subset of members; identifying a member with influence over the subset of members as an identified member, wherein the identified member is identified based on the analyzed communications and one or more influencing factors; determining at least one intervention for the subset of members; providing information about at least one intervention that is beneficial to the subset of members to the identified member; and
providing an incentive to the identified member to use the at least one intervention.

Any of the aspects herein, wherein identifying the intervention includes: determining a number of members of the subset of members that the at least one intervention is applicable to; and selecting the at least one intervention when the number of members is greater than a predetermined threshold.

Any of the aspects herein, wherein identifying the intervention includes: determining a return on investment (ROI) of applying the at least one intervention to the subset of members; and selecting the at least one intervention when the ROI is greater than a predetermined threshold.

Any of the aspects herein, further comprising determining that the at least one intervention is safe for the identified user.

Any of the aspects herein, wherein the subset of members and the identified member are further identified based on member information.

Any of the aspects herein, wherein the at least one intervention is at least one of: a pharmaceutical intervention, a medical procedure, a medical device, drugs, medical surgeries, dietary supplements, wearable sensors, and a lifestyle intervention.

Any of the aspects herein, wherein the lifestyle intervention comprises at least one of: fasting, stress reduction. improving sleep behaviors, improving mood, reducing anxiety, improving relationships and dietary changes.

Any of the aspects herein, wherein the method is used to manage the health care cost of the population.

Any of the aspects herein, wherein the one or more influencing factors comprise a number of connections of the member with the subset of members and a number of interactions between the member and the subset of members.

A method for providing digital communications of an intervention according to at least one embodiment of the present disclosure comprises providing a digital communication network (DCN) for members of a population to communicate with each other; analyzing a first set of communications in the DCN between the members; identifying a subset of members; identifying a member with influence over the subset of members as an identified member, wherein identifying the member includes determining a number of connections of the member with the subset of members and a number of interactions between the member and the subset of members; providing information about an intervention that is beneficial to the subset of members to the identified member and information about an incentive if the identified member shares their experience with the intervention; analyzing a second set of communications in the DCN between the identified member and the subset of members; determining, based on the analysis, that the identified member shared their experience with the intervention to the subset of members; and providing, in response to determining that the identified member shared their experience, the incentive to the identified member.

Any of the aspects herein, wherein the incentive includes providing a means to experience the intervention at a reduced cost.

Any of the aspects herein, wherein the incentive comprises engaging in a behavior and sharing the experience regarding the behavior.

Any of the aspects herein, wherein the behavior is an intervention.

Any of the aspects herein, wherein the intervention is at least one of a pharmaceutical intervention.

Any of the aspects herein, wherein the intervention is a lifestyle intervention.

Any of the aspects herein, wherein the lifestyle intervention comprises at least one of: fasting, stress reduction. improving sleep behaviors, improving mood, reducing anxiety, improving relationships and dietary changes.

Any of the aspects herein, wherein the incentive includes providing a discount or at no cost, means for measuring a biometric of the identified member.

Any of the aspects herein, wherein the biometric is at least one of: a saliva sample, a blood sample, a breath sample and a stool sample.

Any of the aspects herein, further comprising: requesting a biometric sample kit through the DCN.

A system to provide an intervention in a subset of a population according to at least one embodiment of the present disclosure comprises a computer processor; a data repository in communication with the computer processor and storing: communications, an intervention, and an incentive; a communication analyzer which, when executed by the computer processor, analyzes the communications; a digital communications network which, when executed by the computer processor, provides a network for members of a population to interact with each other; and a server controller which, when executed by the computer processor: identifies a subset of members; identifies a member with influence over the subset of members as an identified member; determines at least one intervention for the subset of members; provide information to the identified member about at least one intervention that is beneficial to the subset of members; and provide an incentive to the identified member to use the at least one intervention.

Any of the aspects herein, wherein identifying the identified member includes determining a number of connections of the member with the subset of member and a number of interactions between the member and the subset of members.

### BRIEF DESCRIPTION OF THE DRAWINGS

Aspects of the described embodiments are more evident in the following description, when read in conjunction with the attached Figures.

FIG. 1 shows a simplified diagram of devices, in accordance with one or more embodiments.

FIG. 2 is a logic flow diagram that illustrates the operation of a method, in accordance with one or more embodiments.

FIG. 3 is another logic flow diagram that illustrates the operation of a method, in accordance with one or more embodiments.

FIG. 4A shows an example of a computing system, in accordance with one or more embodiments.

FIG. 4B shows an example of a network, in accordance with one or more embodiments.

### DETAILED DESCRIPTION

Various embodiments are directed to incentivizing health related activity based on communications within a digital communication network (DCN). This may be based in part on creating greater awareness of lifestyle changes and medial options within the DCN. A lifestyle (or behavior) change can be made easier by making it a social process instead of an individual process.

By way of background, as a social process, value can be placed on those members of a community or population that have experience over expertise. Such value can be used to create an atmosphere or community where members can learn about others who have undergone the same interventions that an individual member is contemplating or in the process of doing. Additionally, the community can be used to help support the individual member, which can be a patient under the care of a physician or simply someone interested in the condition and/or disease.

The community can also be used to encourage lifestyle interventions, which are inherently safe. Such community may use the philosophy that any action now is preferred to a “better” action later and also support the concept that ideas and communication are healthcare. Conventionally, lifestyle change has been looked at as an individual pursuit, such as plans personalized just for the individual. Further, medicine is typically a one-on-one activity (reinforced by the privacy concepts the system is based on). In the present disclosure, lifestyle change is seen as highly driven by social parameters and the impact on social parameters is critical, as will be discussed below.

Conventional lifestyle applications may tell individuals the “right thing to do”, which could be right, but given the complexity of the lifestyle change, is likely not to occur. Often, if the lifestyle change suggested works, they can make the individual more dependent on things outside the individual's control. On the other hand, communities share experiences, not expertise, which individuals can try and if they work for them, is a success. In some cases, success can range from slowing the progression of adverse conditions to managing a disease, or the overall risk level in a population.

Lifestyle change may be supported by communities that provide support, ideas, and, in the case of these ideas, access to tools to provide objective data to make meaningful lifestyle changes in an individual user or members of a population. For example, in a community where a person is a peer, the actions they take and learn from are that their volition and may result in increased agency (or autonomy) or self-efficacy. This not only increase the chances of continuous lifestyle improvement, but improved outcomes throughout the heath system.

Online communities can be provided so that people can learn about healthy lifestyle practices and work to improve their health between clinical touchpoints, such as office visits (whether through communications from an influential person or from other members). To help incentivize healthy behaviors, patients can use tools like in-home tests and biosensors that measure how well their health actions are working.

Thus, it is desirable to provide a community where a member with influence over a subset of members can be identified and provided incentives for sharing an intervention and/or their experience with the intervention that is beneficial to the subset of members. Such community can be implemented in a digital communication network (DCN) where members can interact with each other and the DCN. As one example, the community encourages individuals to share what they are doing and how it impacted their condition. A member with influence over other members may also share their experience, which may encourage the other members to try the same experience. The digital communication networks can also manage a member's communication feed to prioritize messages from the member with influence over a subset of members.

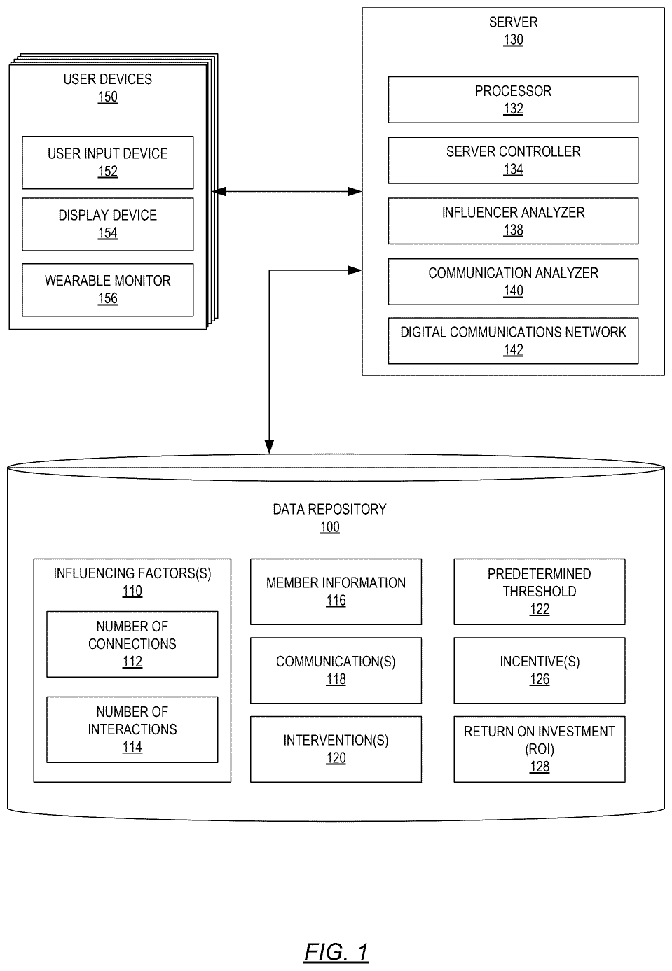

Turning to the Figures, a system is shown in FIG. 1. The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores influencing factors (110). The influencing factors (110) can be used to determine if an identified member has influence over a subset of members. In other words, the influencing factors (110) correlate to whether the subset of members would be influenced by the identified member. For example, if the identified member provides a communication about their experience with, for example a new yoga studio and some of the subset of members then attend the new yoga studio, the identified member can be considered influential over the subset of members.

The influencing factors (110) include factors such as, for example, a number of connections (112) between a member and one or more other members and a number of interactions (114) between the member and one or more other members. The influencing factors (110) can include more or less factors, including factors not described herein.

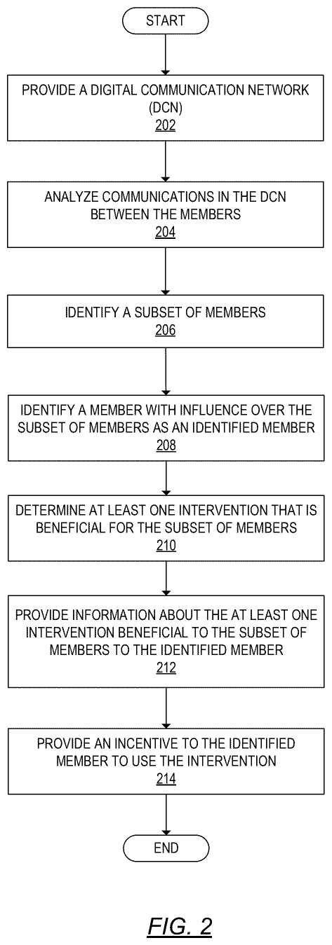

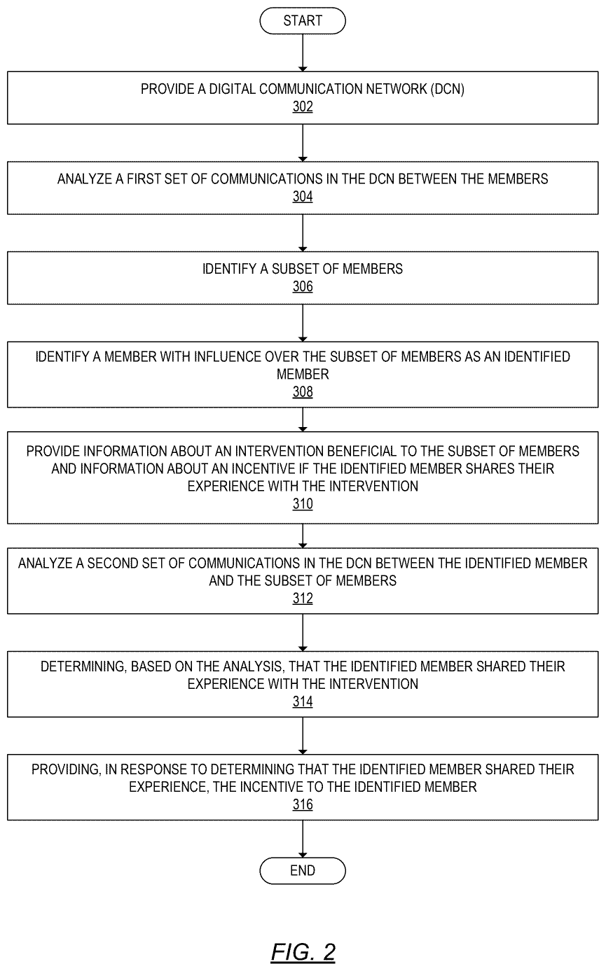

The data repository (100) also stores member information (116). The member information (116) can be used to determine a subset of members and the identified member that has influence over the subset of members. The determination and identification of the identified member and the subset of members are described in detail in FIGS. 2 and 3.

The member information (116) includes information about the identified member that is useful for determining if the identified member is influential over the subset of members. The member information (116) may include information such as whether the identified member is an ambassador or influencer for one or more products or brands, sub-groups that the identified member is a member of, activities that the identified member participates in, demographics of the identified member, etc.

The member information (116) also includes information about each member of the population that may be useful in identifying the member as part of the subgroup of members over which the identified member has influence. The member information (116) may include information such as, for example, groups that each member is a part of, activities each member participates in, demographics of each member, etc.

The data repository (100) also stores communications (118). The communications (118) are communications from members in the DCN (142) or from the DCN (142). The communications may be, for example, text-based, image-based, multimedia communications, audio communications, or any combinations thereof. The communications (118) may be between members, communications in a forum, communications with the DCN (142), or communications generated by the DCN (142) (e.g., communications from a chabot). The communications (118) can include information about a member's experience with a particular intervention (120) and whether the intervention (120) was helpful to the member.

The data repository (100) also stores the interventions (120). The intervention (120) is an activity, suggestion, or recommendation provided to one or more members with the purpose of improving the one or more member's health. The intervention (120) may also be a health-based activity. Such suggestions or activities may include, for example, lifestyle interventions such as fasting, stress reduction, improving sleep behaviors, improving mood, reducing anxiety, improving relationships, participating in a cardio activity, and/or dietary changes. The interventions (120) may also be a medical intervention such as, for example, prescribing medication or pharmaceuticals to the one or more members. In still other embodiments, the intervention (120) may be a medical procedure, a medical device, a medical surgery, dietary supplements, or a wearable device or sensor. In some instances, the intervention (120) may be provided to a member's healthcare provider.

The data repository (100) also stores a predetermined threshold (122). The predetermined threshold (122) is a numerical number, percentage, proportion, or binary question that can be used in determining the intervention (120). The predetermined threshold (122) can be determined by a user such as a developer of the DCN (142). Alternatively, the predetermined threshold (122) can be determined automatically using artificial intelligence, and may thereafter be reviewed and approved (or modified) by the developer or other user.

The data repository (100) also stores incentives (126). The incentives (126) are discounts, coupons, money, or other rewards to incentivize an identified member that has influence over a subset of members to post about the intervention (120) and/or their experience with the intervention (120). The incentives (126) may be, for example, a discount for the intervention (120). For example, the intervention (120) may be attending a yoga class, and the incentive (126) may be a coupon to cover the cost of the yoga class for the identified user. The incentive (126) may include one or more incentives (126).

In some embodiments, the incentive (126) may include providing a discount or at no cost, means for measuring a biometric of the identified member. The biometric may be obtained from a saliva sample, a blood sample, a breath sample, or a stool sample. A biometric kit may be obtained through the DCN (142), a clinic, or other source. Biometrics are a useful incentive as biometrics can be used in many instances such as the provisioning and commissioning of healthcare. For example, biometrics can be used to diagnose diabetes, prescribe hypertension medication with a patient's blood pressure exceeds a certain value, prescribe cancer drugs, etc.

The data repository (100) also stores a return on investment (ROI) (128). The ROI (128) is an estimated return on investment of one or more members of the subset of members using or adopting the intervention (120). More specifically, the ROI (128) may be a savings in healthcare costs for the one or more members or the population as whole. For example, the ROI (128) may be the savings in costs of a member no longer using a medication after adopting the intervention (120). Factors such as, for example, a member's spending or purchases (or reduction thereof) of health-related medications or aids, the number of visit to a physician by the member, etc. may be used to calculate the ROI (128).

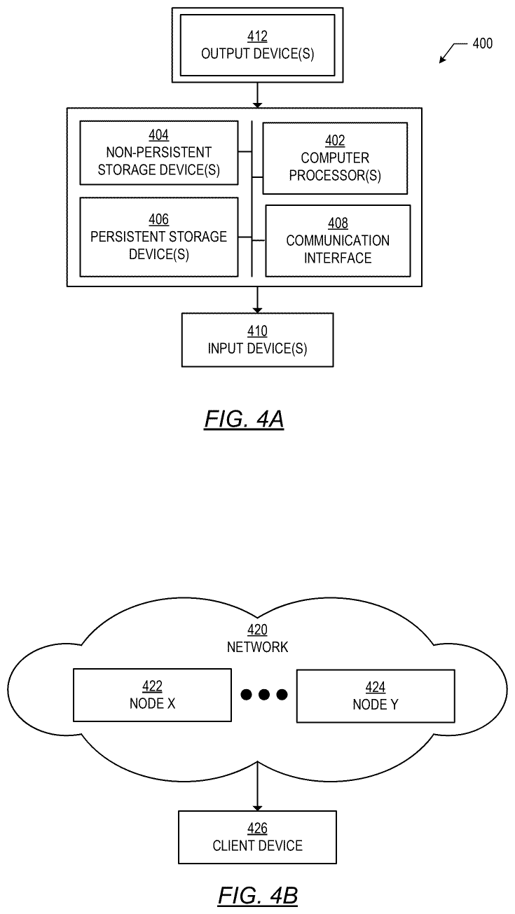

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as an influencer analyzer (138) or a communication analyzer (140). An example of a computer system and network that may form the server (130) is described with respect to FIG. 4A and FIG. 4B.

The server (130) also includes a processor (132). The processor (132) is one or more hardware or virtual processors which may execute computer readable program code that defines one or more applications, such as the influencer analyzer (138) or the stress communication analyzer (140). An example of the processor (132) is described with respect to the computer processor(s) (402) of FIG. 4A.

The server (130) also may include a server controller (134). The server controller (134) is software or application specific hardware which, when executed by the computer processor (132), controls and coordinates operation of the software or application specific hardware described herein. Thus, the sever controller (134) may control and coordinate execution of the influencer analyzer (138) or the stress communication analyzer (140).

The server (130) also includes the influencer analyzer (138). The influencer analyzer (138) is software or application specific hardware which, when executed by the computer processor (132) receives at least the member information (116) and the influencing factors (110) as input to determine an identified member that has influence over a subset of members. The influencer analyzer (128) may also receive the communications (118) for determining the identified member.

The server (130) also includes a communication analyzer (140). The communication processor (140) is software or application specific hardware which, when executed by the computer processor (132), receives the communications (118) as input and determines the subset of members. The communication analyzer (140) may also prioritize the communications (118) presented to the subset of members. For example, communications (118) from the identified member regarding the intervention (120) and/or the identified member's experience with the intervention (120) may be prioritized higher.

The server (130) also includes the digital communications network (DCN) (142). The DCN (142) is a network through which members of a population can interact with each other, or with a system supported by the DCN (142). The DCN (142) can also foster a community in which some members may have influence over other members via, for example, forums, chat room, social media, etc. In such embodiments, the DCN (142) can be used in determining and identifying an identified member that has influence over a subset of members. The DCN (142) can also be used by the identified member to post, share, or otherwise communicate the intervention (120) to the subset of members or to the entire population of members.

In the DCN (142), communications (118) can be prioritized to more highly prioritize communications from the identified member. These communications (118) between members of the population can be one-to-one, one-to-many, one-to-system, system-to-one, or system-to-many. The system may also feature an AI bot that derives its communications from analysis of communications in the digital communication network and/or biometrics provided to the DCN (142).

The system shown in FIG. I also may include one or more user devices (150). The user devices (150) may be considered remote or local. A remote user device is a device operated by a third-party (e.g., an end user of a chabot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (400) shown in FIG. 4A) that communicate with the server (130). The user devices (150) may include a wearable monitor (156) and be configured to send stress indicator data (110) to the server (130). In an alternative embodiment, a separate wearable device or monitor (156) may be in communication with the user device (150), such as a smart watch, or blood pressure monitor. The user devices (150) may also include a user input device (152) and/or a display device (154).

While FIG. 1 shows a configuration of components, other configurations may be used without departing from the scope of one or more embodiments. For example, various components may be combined to create a single component. As another example, the functionality performed by a single component may be performed by two or more components.

FIG. 2 is a logic flow diagram that illustrates a method, and a result of execution of computer program instructions, in accordance with various embodiments. The method of FIG. 2 may be used to identify a member that has influence over a subset of members and incentivize the identified member to communicate an intervention to the subset of members.

At Step 202, a digital communication network (DCN) is provided. The DCN may be the same as or similar to the DCN (142) and provides a network for members of a population to interact with each other or with the DCN (142).

At Step 204, communications in the DCN between the members are analyzed. The communications may be the same as or similar to the communications (118) and may be analyzed by a communication analyzer such as the communication analyzer (140). The communication analyzer may also receive member information such as the member information (116). The communication analyzer may analyze the communications to determine a subset of members of the population.

At Step 206, the subset of members is identified. In some embodiments, the subset of members is a group of members that is less than the entire population of members. In other embodiments, the subset of members may be the entire population. The subset of members is identified by the communication analyzer using the member information and communications. The subset of members may be identified by members that are part of the same groups (e.g., social groups, community groups, etc.), by demographics, by having the same medical condition or disease, or any combination thereof. The subset of members may also be identified based on communications between the members. For example, the subset of members may all have communicated with each other or participated in the same forum or chat room about a particular subject.

At Step 208, a member with influence over the subset of members is identified as an identified member. It will be appreciated that the identified member can include one identified member, two identified members, or more than two identified members.

The identified member is identified by the influencer analyzer using the member information, the communications, and/or the influencing factors. The identified member may be identified based on a number of connections that the identified member has with the subset of members and/or a number of interactions that the identified member has had with member(s) of the subset of members. The identified member may also be identified based on the member information. For example, the identified member may have shared demographics, medical conditions, disease, activities, common interests, etc. with at least some or all of the subset of members.

In some embodiments, the identified member may be identified prior to identifying the subset of members. In such embodiments, the identified member may be used to identify the subset of members. For example, other members that already follow, communicate, or are otherwise connected to the identified member may be identified as the subset of members.

At Step 210, at least one intervention is determined for the subset of members. The intervention may be determined using medical guidelines, the communications in the DCN, health claims by members in the population, and the member information.

In at least one embodiment, the at least one intervention may be determined by determining a number of members of the subset of members that the intervention is applicable to and selecting the intervention when the number of members is greater than a predetermined threshold such as the predetermined threshold (122). For example, the number of members may each have chronic stress and the intervention applicable to reducing stress may be yoga. If the number of members that have chronic stress is greater than the predetermined threshold, then the intervention of yoga will be selected.

In another embodiment, the intervention may be determined by determining an ROI such as the ROI (128) of applying the at least one intervention to the subset of members and selecting the intervention when the ROI is greater than the predetermined threshold. For example, the predetermined threshold may be greater than 50% ROI and the ROI may be 70% when the intervention is yoga and 40% when the intervention is meditation. Thus, yoga is identified as the intervention in such embodiments.

At Step 212, information about the at least one intervention that is beneficial to the subset of members is provided to the identified member. The information may be transmitted to a user device such as the user device (150) by the DCN. The information may include, for example, a time or place where the identified member can participate in the at least one intervention. The information may also include instructions for accessing the at least one intervention. For example, the at least one intervention may be a yoga class and the information may include a yoga studio near the identified member, times for one or more yoga classes, and information about signing up for a yoga class.

In some embodiments, the intervention may have a low ROI for the identified member, but may have a high ROI for the subset of members. In other words, the intervention may be deemed appropriate, but not necessary for the identified member. In such embodiments, the intervention may still be provided to the identified member as the intervention may be helpful to the subset of members. Further, though the intervention may be less valuable to the identified member, the identified member providing the intervention may provide a high value to the subset of members.

At Step 214, an incentive is provided to the identified member. The incentive may be the same as or similar to the incentive (126) and may be delivered to the identified member via the DCN, mail, or may be communicated to a provider of the intervention. The incentive may be provided to the identified member prior to, during, and/or after the identified member has shared or communication the intervention to the subset of members. In some embodiments, the incentive is provided after verification that the identified member has participated in and/or communicated their experience with the incentive, as described in FIG. 3 below.

In some embodiments, the incentive provided may be based on past reactions to an incentive. For example, an identified member who responded well to prior incentives may be provided further options. Alternatively, an identified member who did not respond to an early incentive may be offered an additional or greater incentive to engage with the intervention.

The method of FIG. 2 may include other steps such as determining that the intervention is safe for the identified member. For example, if the intervention is jogging, it may be determined that the identified user is injury free and can participate in jogging. Such determination prevents an inappropriate intervention being sent to the identified user. In some embodiments, an intervention may not be provided to the identified member if the intervention is deemed inappropriate and unnecessary for the identified member.

In still other embodiments, the method of FIG. 2 may also include identifying the identified member to a primary care provider. The primary care provider can then use this information to, for example, align their treatment with interventions communicated by the identified member. In another example, the primary care provider may request to co-communicate an intervention with the identified member.

The method of FIG. 2 may also include steps in which an efficacy of the intervention is measured. In such embodiments, a first set of biometrics may be obtained from the subset of members prior to the identified member communicating the intervention and a second set of biometrics may be obtained from the subset of members after the identified member has communicated the intervention to the subset of members. The difference between the first set of biometrics and the second set of biometrics can be used to determine whether the intervention is effective and working.

The method of FIG. 2 described above may have more or less steps than shown. Further, steps may be repeated as needed. For example, the Steps 210, 212, 214 may be repeated to identify and provide new interventions for the subset of members. In another example, the Steps 204, 206, 207 may be repeated to adjust the subset of members or to identify a new identified member.

FIG. 3 is a logic flow diagram that illustrates a method, and a result of execution of computer program instructions, in accordance with various embodiments. The method can be used to identify an identified member that has influence over a subset of members and to verify that the identified member has communicated an intervention to the subset of members prior to providing an incentive to the identified member.

At Step 302, a digital communications network (DCN) is provided. The Step 302 is the same as or similar to the Step 202 of FIG. 2 above.

At Step 304, a first set of communications is analyzed in the DCN between members. The Step 304 is the same as or similar to the Step 204 of FIG. 2 above.

At Step 306, a subset of members is identified. The Step 306 is the same as or similar to the Step 206 of FIG. 2 above.

At Step 308, a member with influence over the subset of members is identified as an identified member. The Step 308 is the same as or similar to the Step 208 of FIG. 2 above. It will be appreciated that the identified member can include one identified member, two identified members, or more than two identified members.

At Step 310, information about an intervention that is beneficial to the subset of members is provided to the identified member and information about an incentive is provided to the identified member. The information about the intervention may include, for example, a time or place where the identified member can participate in the at least one intervention. The information may also include instructions for accessing the at least one intervention. For example, the at least one intervention may be a yoga class and the information may include a yoga studio near the identified member, times for one or more yoga classes, and information about signing up for a yoga class. The information about the incentive may include, for example, that the identified member must participate in the intervention a minimum number of times and must communicate their experience with the intervention to the subset of members before being provided with the incentive.

At Step 312, a second set of communications between the subset of members and the identified member is analyzed. The Step 312 may be the same as or similar to the Step 204 of FIG. 2 above, except that the second set of communications is analyzed to determine if the identified member has shared or communicated their experience with the intervention.

At Step 314, based on the analysis, the identified member is determined to have shared their experience with the intervention. In at least one embodiment, the shared experience may be determined based on a keyword search of communications from the identified member to the subset of members. For example, if the intervention is yoga, the communications may be searched for “yoga” and other key words indicating the identified member's experience such as “attended”, “enjoyed”, “excited”, etc.

At Step 316, an incentive is provided to the identified member in response to determining that the identified member shared their experience. The Step 316 may be the same as or similar to the Step 214 of FIG. 2 described above, except that the incentive is provided after the promotion of the intervention and not prior to or during the identified member's promotion and/or participation in the intervention.

The method of FIG. 3 described above may have more or less steps than shown. Further, steps may be repeated as needed. For example, the Step 312 may be repeated until the identified member has communicated their experience with the intervention.

The methods of FIGS. 2 and 3 can be used to, for example, manage a health care cost of the population. By promoting healthy lifestyles by encouraging influential members to participate in and share their experience with a health-based incentive, members of the population may use less health care after adopting healthy habits and thus, may decrease the cost of health care for the population. In other embodiments, the targeting of identified members may be done to assist in the slowing of the progression of an adverse health condition or managing a disease in an individual member or the population.

In still other embodiments, the method may be used to slow the progression of adverse metabolic conditions in an individual member of a population. The adverse metabolic condition can be for example, chronic systemic inflammation (CSI); malaise or low-energy; and/or a disease. The disease may be a prodromal disease, Obesity, Diabetes, rheumatoid arthritis, Crohn's, Psoriasis, eczema, cardiovascular disease, congestive heart failure, chronic obstructive pulmonary disease, asthma, depression, or anxiety. In some cases, the condition may be social dysfunction.

A specific example of FIGS. 2 and 3 will now be described. In such example, the DCN includes a social media network in which a population of members post communications to the network and post communications to each other. At least a first set of communications including communications to the network and communications between members are analyzed to determine a subset of members with at least one commonality. The at least one commonality may be, for example, chronic stress. A subset of members is identified that includes such members with chronic stress, as determined by the communications and/or information about the members themselves.

The first set of communications are also used to identify a member that has influence over the subset of members. The identified member may be part of the subset of members, though the identified member need not be part of the subset of members. The identified member may be identified based on the identified member having a connection to a majority of the subset of members. For example, the identified member may have interacted directly with the majority of members or may have influenced some members to participate in an activity with the identified member.

Once the identified member is identified, an intervention that is beneficial to the subset of members is identified. In such example, the intervention may be yoga or meditation to target the chronic stress of the subset of members. Instructions are then sent to the identified member to share or communicate the intervention itself and/or their personal experience with the intervention. Thus, the identified member is provided information about yoga or meditation and encouraged to share their own personal experience with yoga or meditation and how it has impacted their life with the subset of members. The identified member may communicate about yoga or meditation (and/or their experience) using a text-based post, posting a video or audio of their experience with yoga or meditation, and/or an image in combination with text, video, and/or audio. The communication provided by the identified member may be prioritized over other communications in the social media network at large, or specifically to the subset of members.

A second set of communications may be analyzed to validate that the identified member has communicated the intervention and/or their experience with the intervention. The second set of communications may be communications from the identified member to the subset of members or communications from the identified member to forums, the social media network, etc. in which the subset of members may see the communications. Once the identified member's communication has been validated, an incentive may be provided to the identified member for communicating about the intervention. The incentive may be, for example, a free yoga class or meditation session.

The above example exemplifies how interventions that promote healthy lifestyles and habits can be disseminated within a grouping of members who would most benefit from the intervention. While members of the subgroup may ignore or disregard instructions from (for example) a clinician to participate in an intervention such as yoga, the same members may be influenced or convinced to try yoga from another member. The members may find the influential members more personal to themselves, and may be more likely to adopt or participate in the intervention.

Other embodiments of the methods describe above are now provided.

In at least one embodiment, a method is further provided to manage health care risk in a population. The method includes providing the means for members of a population to communicate with each other through the DCN. Members of the population are informed about incentives for sharing their experience or their reflection on their experience with a self-selected behavior. When the DCN automatically observes the sharing of experiences or reflections in the online social network, it can pay the member of the population the stated incentive for their sharing of experiences and reflections.

In another embodiment, risks contracts providers often provide incentives for engaging in certain behaviors that improve their health or reduce the risk (or cost) of a future event. These include engaging with the medical system in various ways, such as, to get a colonoscopy, or an annual physical; or lifestyle behaviors, such as, walk 10k steps a day. Documentation of completion of the health improving behavior may be used to authorize the awarding of incentives. This can be used to reduce risk in a population by providing incentives to share reflections on health improving behaviors in an online DCN.

Similar to providing incentives for sharing of experiences, various embodiments include providing incentives for providing samples for tests. The samples may not be used to provide information useful in comparing the results to a standard use to make medical decisions; instead, the results can be used for the member to learn about themselves and to manage health care risk in a population.

The method includes providing the means for members of said population to communicate with each other through a DCN. The DCN can be used to inform the members of the population about incentives for providing samples for testing related to their lifestyle behaviors. Members of the population are allowed to order sampling kits for said test, for example, through the DCN. The DCN can then have the member of the population a sample for testing. The results of the test are provided by the DCN to the members the population providing the sample, and the DCN can automatically grant the member of the population the stated incentive (for example, send them a payment) for providing the sample based on their interactions with the community.

In some embodiments, the test results are compared a previous sample provided rather than with a standard used to diagnose or treat a disease. The biological sample can be a saliva sample. a blood sample, and/or a stool sample. The sample may be collected provided at home. The DCN may send a sample collection kit to the member. In some embodiments, the behavior is providing a survey or psychological instrument.

The incentive can be based on the member of the population sharing their experience or reflections concerning providing a sample or thinking about the result. The member may be asked to share multiple times. Providing incentives for behaviors or learning about oneself is useful and can be a more powerful technique to encourage deeper reflection and promote agency building.

Another embodiment provides a method to manage health care risk in a population. The method includes providing the means for members of said population to communicate with each other through the DCN. Members of the population are informed of incentives for engaging in self-selected health improving behaviors and provided samples for testing related to their self-selected lifestyle behaviors. The members of the population are allowed to order sampling kits for said test (for example, through the DCN). The member of the population can then provide two of more samples before, during, or after engaging in the behavior. The results of the test(s) are reported to the members the population providing the sample, and the DCN can automatically pay the member of the population the stated incentive for providing the sample based on their interactions with the community.

The communications of available incentives may be provided through an online social network. The communications between members of the population may be one-to-one, one-to-many, one-to-system, system-to-one, or system-to-many.

The incentive can include participating in behavior. The behavior may be a healthy lifestyle behavior; an interaction with the medical system; the use of a drug or supplement or medical device; and/or providing a biological sample. The biological sample can be a saliva sample. a blood sample, and/or a stool sample. The sample may be collected provided at home. The DCN may send a sample collection kit to the member. In some embodiments, the behavior is providing a survey or psychological instrument.

The incentive may be based on having the member perform multiple occurrences of sharing of reflections and/or experiences. The incentive can be based on the member of the population sharing their experience or reflections concerning providing a sample or thinking about the result. The member may be asked to share multiple times. In some embodiments, the method may be done to assist in the slowing of the progression of an adverse health condition or managing a disease.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 4A, the computing system (400) may include one or more computer processor(s) (402), non-persistent storage device(s) (404), persistent storage device(s) (406), a communication interface (408) (e.g., Bluetooth interface, infrared interface, network interface, optical interface, etc.), and numerous other elements and functionalities that implement the features and elements of the disclosure. The computer processor(s) (402) may be an integrated circuit for processing instructions. The computer processor(s) (402) may be one or more cores, or micro-cores, of a processor. The computer processor(s) (402) includes one or more processors. The computer processor(s) (402) may include a central processing unit (CPU), a graphics processing unit (GPU), a tensor processing unit (TPU), combinations thereof, etc.

The input device(s) (410) may include a touchscreen, keyboard, mouse, microphone, touchpad, electronic pen, or any other type of input device. The input device(s) (410) may receive inputs from a user that are responsive to data and messages presented by the output device(s) (412). The inputs may include text input, audio input, video input, etc., which may be processed and transmitted by the computing system (400) in accordance with one or more embodiments. The communication interface (408) may include an integrated circuit for connecting the computing system (400) to a network (not shown) (e.g., a local area network (LAN), a wide area network (WAN) such as the Internet, mobile network, or any other type of network) or to another device, such as another computing device, and combinations thereof.

Further, the output device(s) (412) may include a display device, a printer, external storage, or any other output device. One or more of the output device(s) (412) may be the same or different from the input device(s) (410). The input device(s) (410) and output device(s) (412) may be locally or remotely connected to the computer processor(s) (402). Many different types of computing systems exist, and the aforementioned input device(s) (410) and output device(s) (412) may take other forms. The output device(s) (412) may display data and messages that are transmitted and received by the computing system (400). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (402), is configured to perform one or more embodiments, which may include transmitting, receiving, presenting, and displaying data and messages described in the other figures of the disclosure.

The computing system (400) in FIG. 4A may be connected to, or be a part of, a network. For example, as shown in FIG. 4B, the network (420) may include multiple nodes (e.g., node X (422) and node Y (424), as well as extant intervening nodes between node X (422) and node Y (424)). Each node may correspond to a computing system, such as the computing system shown in FIG. 4A, or a group of nodes combined may correspond to the computing system shown in FIG. 4A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (400) may be located at a remote location and connected to the other elements over a network.

The nodes (e.g., node X (422) and node Y (424)) in the network (420) may be configured to provide services for a client device (426). The services may include receiving requests and transmitting responses to the client device (426). For example, the nodes may be part of a cloud computing system. The client device (426) may be a computing system, such as the computing system shown in FIG. 4A. Further, the client device (426) may include or perform all or a portion of one or more embodiments.

The computing system of FIG. 4A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method to provide an intervention in a subset of a population, the method comprising:
providing a digital communication network (DCN) for members of a population to communicate with each other;
analyzing communications in the DCN between the members;
identifying the subset of members;
identifying a member with influence over the subset of members as an identified member, wherein the identified member is identified based on the analyzed communications and one or more influencing factors;
determining at least one intervention for the subset of members;
providing information about at least one intervention that is beneficial to the subset of members to the identified member; and
providing an incentive to the identified member to use the at least one intervention.

2. The method of claim 1, wherein identifying the intervention includes:
determining a number of members of the subset of members that the at least one intervention is applicable to; and
selecting the at least one intervention when the number of members is greater than a predetermined threshold.

3. The method of claim 1, wherein identifying the intervention includes:
determining a return on investment (ROI) of applying the at least one intervention to the subset of members; and
selecting the at least one intervention when the ROI is greater than a predetermined threshold.

4. The method of claim 1, further comprising determining that the at least one intervention is safe for the identified user.

5. The method of claim 1, wherein the subset of members and the identified member are further identified based on member information.

6. The method of claim 1, wherein the at least one intervention is at least one of: a pharmaceutical intervention, a medical procedure, a medical device, drugs, medical surgeries, dietary supplements, wearable sensors, and a lifestyle intervention.

7. The method of claim 6, wherein the lifestyle intervention comprises at least one of: fasting, stress reduction. improving sleep behaviors, improving mood, reducing anxiety, improving relationships and dietary changes.

8. The method of claim 1, wherein the method is used to manage the health care cost of the population.

9. The method of claim 1, wherein the one or more influencing factors comprise a number of connections of the member with the subset of members and a number of interactions between the member and the subset of members.

10. A method for providing digital communications of an intervention, the method comprising:
providing a digital communication network (DCN) for members of a population to communicate with each other;
analyzing a first set of communications in the DCN between the members;
identifying a subset of members;
identifying a member with influence over the subset of members as an identified member, wherein identifying the member includes determining a number of connections of the member with the subset of members and a number of interactions between the member and the subset of members;
providing information about an intervention that is beneficial to the subset of members to the identified member and information about an incentive if the identified member shares their experience with the intervention;
analyzing a second set of communications in the DCN between the identified member and the subset of members;
determining, based on the analysis, that the identified member shared their experience with the intervention to the subset of members; and
providing, in response to determining that the identified member shared their experience, the incentive to the identified member.

11. The method of claim 10, wherein the incentive includes providing a means to experience the intervention at a reduced cost.

12. The method of claim 10, wherein the incentive comprises engaging in a behavior and sharing the experience regarding the behavior.

13. The method of claim 12, wherein the behavior is an intervention.

14. The method of claim 13, wherein the intervention is at least one of a pharmaceutical intervention.

15. The method of claim 13, wherein the intervention is a lifestyle intervention.

16. The method of claim 15, wherein the lifestyle intervention comprises at least one of: fasting, stress reduction. improving sleep behaviors, improving mood, reducing anxiety, improving relationships and dietary changes.

17. The method of claim 10, wherein the incentive includes providing a discount or at no cost, means for measuring a biometric of the identified member.

18. The method of claim 17, wherein the biometric is at least one of: a saliva sample, a blood sample, a breath sample and a stool sample.

19. The method of claim 17, further comprising:
requesting a biometric sample kit through the DCN.

20. A system to provide an intervention in a subset of a population, the system comprising:
a computer processor;
a data repository in communication with the computer processor and storing:
communications,
an intervention, and
an incentive;
a communication analyzer which, when executed by the computer processor, analyzes the communications;
a digital communications network which, when executed by the computer processor, provides a network for members of a population to interact with each other; and
a server controller which, when executed by the computer processor:
identifies a subset of members;
identifies a member with influence over the subset of members as an identified member;
determines at least one intervention for the subset of members;
provide information to the identified member about at least one intervention that is beneficial to the subset of members; and
provide an incentive to the identified member to use the at least one intervention.

21. The system of claim 20, wherein identifying the identified member includes determining a number of connections of the member with the subset of member and a number of interactions between the member and the subset of members.
